SEQUENCE LISTING (34 sequences)

SEQ ID NO: 1 - DNA, 30 bp, Artificial Sequence
  Fully-synthetic aptamer for hepatitis C virus (HCV)
acgggataccggcgcttgatttagctcggg

SEQ ID NO: 2 - DNA, 30 bp, Artificial Sequence
  Fully-synthetic aptamer for hepatitis C virus (HCV)
acagacacaggtgctactgggaactagatc

SEQ ID NO: 3 - DNA, 34 bp, Artificial Sequence
  Fully-synthetic aptamer for hepatitis C virus (HCV)
actaatacgtgacggggagatgcatcgtgttgtc

SEQ ID NO: 4 - DNA, 30 bp, Artificial Sequence
  Fully-synthetic aptamer for hepatitis C virus (HCV)
ggttcgtcgggatgaggcaagatttggctc

SEQ ID NO: 5 - DNA, 30 bp, Artificial Sequence
  Fully-synthetic aptamer for hepatitis C virus (HCV)
taatgtgaaataagctagctcaccggctga

SEQ ID NO: 6 - DNA, 30 bp, Artificial Sequence
  Fully-synthetic aptamer for hepatitis C virus (HCV)
agttcgactccgttaggtgtcgctgtaggt

SEQ ID NO: 7 - DNA, 30 bp, Artificial Sequence
  Fully-synthetic aptamer for hepatitis C virus (HCV)
tacatgcggagattaattggtcctacggag

SEQ ID NO: 8 - DNA, 30 bp, Artificial Sequence
  Fully-synthetic aptamer for hepatitis C virus (HCV)
tgcatgctgtgattatgggagttagtgggc

SEQ ID NO: 9 - DNA, 29 bp, Artificial Sequence
  Fully-synthetic aptamer for hepatitis C virus (HCV)
taatgtggaataagctagctaccggctga

SEQ ID NO: 10 - DNA, 30 bp, Artificial Sequence
  Fully-synthetic aptamer for hepatitis C virus (HCV)
gagaggagtggtggaagcaggtagtcttat

SEQ ID NO: 11 - DNA, 30 bp, Artificial Sequence
  Fully-synthetic aptamer for hepatitis C virus (HCV)
tgggggggtctggtgagcaaacgtggggag

SEQ ID NO: 12 - DNA, 30 bp, Artificial Sequence
  Fully-synthetic aptamer for hepatitis C virus (HCV)
gtgtacttgcaagctgtttcatagtgcacg

SEQ ID NO: 13 - DNA, 31 bp, Artificial Sequence
  Fully-synthetic aptamer for hepatitis C virus (HCV)
agacgcatggtgttatacatagtgtttgtac

SEQ ID NO: 14 - DNA, 30 bp, Artificial Sequence
  Fully-synthetic aptamer for hepatitis C virus (HCV)
atagggcacttgtctcacaccacggtgtga

SEQ ID NO: 15 - DNA, 30 bp, Artificial Sequence
  Fully-synthetic aptamer for hepatitis C virus (HCV)
gaatcgactataaggacgagacgtgatgtg

SEQ ID NO: 16 - DNA, 30 bp, Artificial Sequence
  Fully-synthetic aptamer for hepatitis C virus (HCV)
tccattcatgtaacgaacatagttttggca

SEQ ID NO: 17 - DNA, 31 bp, Artificial Sequence
  Fully-synthetic aptamer for hepatitis C virus (HCV)
taatgtgaaataatctagcgcacttcgctga

SEQ ID NO: 18 - DNA, 30 bp, Artificial Sequence
  Fully-synthetic aptamer for hepatitis C virus (HCV)
gaatgaggaataatctagctccttcgctga

SEQ ID NO: 19 - DNA, 30 bp, Artificial Sequence
  Fully-synthetic aptamer for hepatitis C virus (HCV)
gaagcactgaggggatgagtagacggcaca

SEQ ID NO: 20 - DNA, 32 bp, Artificial Sequence
  Fully-synthetic aptamer for hepatitis C virus (HCV)
tacggtaggagaggatgttggctcgcgccgtc

SEQ ID NO: 21 - DNA, 30 bp, Artificial Sequence
  Fully-synthetic aptamer for hepatitis C virus (HCV)
aacgagtgtgcatcctcgagtgtttggaca

SEQ ID NO: 22 - DNA, 30 bp, Artificial Sequence
  Fully-synthetic aptamer for hepatitis C virus (HCV)
gtatttgttgtagtgcgagaccaaggttca

SEQ ID NO: 23 - DNA, 30 bp, Artificial Sequence
  Fully-synthetic aptamer for hepatitis C virus (HCV)
aatattttttgcgcgggggtgcgcgaattt

SEQ ID NO: 24 - DNA, 30 bp, Artificial Sequence
  Fully-synthetic aptamer for hepatitis C virus (HCV)
taaaatagaagctccgtaagcagcagtcgg

SEQ ID NO: 25 - DNA, 30 bp, Artificial Sequence
  Fully-synthetic aptamer for hepatitis C virus (HCV)
cgccgtattaagattgggagacctggtaga

SEQ ID NO: 26 - DNA, 30 bp, Artificial Sequence
  Fully-synthetic aptamer for hepatitis C virus (HCV)
gcctgcaggtcgactctagaggatcccgca

SEQ ID NO: 27 - DNA, 26 bp, Artificial Sequence
  Fully-synthetic aptamer for hepatitis C virus (HCV)
tactaatggtttgagcgttcgttgtt

SEQ ID NO: 28 - DNA, 38 bp, Artificial Sequence
  Fully-synthetic aptamer for hepatitis C virus (HCV)
taagtcttgccattagggctgcgtcatgggatcccgca

SEQ ID NO: 29 - DNA, 30 bp, Artificial Sequence
  Fully-synthetic aptamer for hepatitis C virus (HCV)
cgcttctgctgtgtttgggacccgctcgca

SEQ ID NO: 30 - DNA, 88 bp, Artificial Sequence
  Fully-synthetic single-stranded  DNA library
gcggaattctaatacgactcactatagggaacagtccgagccnnnnnnnnnnnnnnnnnnnnnnnnnnnnnngggtcaatgcgtcata

SEQ ID NO: 31 - DNA, 42 bp, Artificial Sequence
  Fully-synthetic upstream primer, comprising EcoRI restriction site
gcggaattctaatacgactcactatagggaacagtccgagcc

SEQ ID NO: 32 - DNA, 25 bp, Artificial Sequence
  Fully-synthetic downstream primer, comprising BamHI restriction site
gcgggatcctatgacgcattgaccc

SEQ ID NO: 33 - DNA, 26 bp, Artificial Sequence
  Fully-synthetic upstream primer for HCV amplification
aatggctcgaggaaactgtgaagcga

SEQ ID NO: 34 - DNA, 26 bp, Artificial Sequence
  Fully-synthetic downstream primer for HCV amplification
ttcatcatgccaatggtgttcgtggc

(19) United States
(12) Patent Application Publication
(10) Pub. No.: US 2010/0184837 A1
(43) Pub. Date: Jul. 22, 2010

(54) Small-molecule nucleotide aptamer for hepatitis C virus, preparation method and use thereof

(76) Inventors: Xiaolian ZHANG, Wuhan (CN); Fang Chen, Wuhan (CN)

Correspondence Address: MATTHIAS SCHOLL, 14781 MEMORIAL DRIVE, SUITE 1319, HOUSTON, TX, 77079, US

(21) Appl. No.: 12/581,930

(22) Filed: Oct. 20, 2009

(30) Foreign Application Priority Data
Oct. 21, 2008 (CN) 200810197315.4

(51) Int. Cl. A61K 31/711; C07H 21/04; C12P 19/34; A61P 31/12

(52) U.S. Cl. 514/44 R; 536/23.1; 435/91.2

(57) ABSTRACT

A DNA aptamer specific for HCV having a nucleotide sequence as shown in SEQIDNO.1-29, and a method of preparing the same including the steps of: (1) constructing a single-stranded DNA library; (2) constructing a double-stranded DNA library; (3) screening by SELEX; (4) amplifying by PCR; (5) cloning and sequencing; and (6) testing the effect from cellular level in vitro. The DNA aptamer can be used directly as medication and diagnostic reagent for detection, prevention, and treatment of hepatitis C. A method for detection of HCV infection is also provided.

### CROSS-REFERENCE TO RELATED APPLICATIONS

Pursuant to 35 U.S.C. §119 and the Paris Convention Treaty, this application claims priority benefits to Chinese Patent Application No. 200810197315.4 filed on Oct. 21, 2008, the contents of which are incorporated herein by reference.

### BACKGROUND OF THE INVENTION

1. Field of the Invention

The invention relates to a small-molecule nucleotide sequence (DNA aptamer) and a method of preparing the same, and more particularly to a small-molecule nucleotide sequence for hepatitis C virus (HCV), preparation method and method of use thereof.

2. Description of the Related Art

Hepatitis C virus (HCV) was firstly identified in 1989. Nowadays, approximately 170 million people worldwide suffer from this infectious disease. In China, the number is about 3.2% of total population, and 80% of acute infections become persistent. More terribly, the infection rate has been increasing with passing day.

The hepatitis C virus (HCV) is mainly spread by blood-to-blood contact. The infection is often asymptomatic, but once established, chronic infection can progress to scarring of the liver (fibrosis), and advanced scarring (cirrhosis). In some cases, those with cirrhosis will go on to develop liver cancer. Clinical treatment of hepatitis C basically depends on anti-virus medication such as IFN-α or IFN-α coupling with ribavirin. However, the treatment has some effect only on those in early infection, and no vaccine against hepatitis C is available to date. Therefore, to detect HCV accurately and sensitively in blood source before transfusion is a key step for prevention of HCV infection.

Now it is clear that HCV is a single-stranded positive RNA-containing member of the flavivirus family, approximately 9.6 kb in length. It contains a single large open reading frame (ORF). The HCV ORF encodes a polypeptide of about 3010 amino acid residues. This polypeptide has been proteolyticaly processed into 9 different structural proteins and non structural proteins by the co-action of proteolytic enzymes of HCV and a host thereof. After the host signal peptide is hydrolyzed, HCV envelope glycoprotein E1 (gp35) and E2 (gp70) come into being. Although the infection and replication mechanism of the virus is not definitely clear from molecular level, the glycoprotein E2 is very important for the virus to adhere to and invade host cells. The glycoprotein E2 adheres to and invades host cells at early infection by recognizing and binding to CD81, a surface receptor of human liver cells.

Nowadays, clinical methods of detecting and diagnosing HCV infection include: (1) enzyme-linked immunosorbent assay (ELISA) to detect antibody against HCV, such as recombinant immunoblot assay (RIBA); (2) RT-PCR to detect HVC RNA, such as fluorescent PCR, immune-PCR(PCR-ELISA), and branch DNA (bDNA) technology; and (3) biochip detection technology to detect HCV gene.

ELISA is easy for practice and has been widely used by blood collection and supply agencies, but the method can not detect HCV from blood samples of patients in window phase (in this phase, a patient has been infected but no antibody produced), and a false positive or false negative result may be obtained due to a series of uncertain factors including but not limited to the sensitivity of kit, the technical proficiency of operators, their sense of responsibility, laboratory temperature, and the quality of sample-adding instrument.

RT-PCR is costly. Although branch DNA (bDNA) technology features high stability, repeatability, and an accurate result, its disadvantages such as low amplification, low sensitivity, narrow detection range, and being not applicable for detecting a low level of HCV RNA are also obvious.

Biochip detection technology is suitable for study of the HCV epidemiology, mutation trend, transmission mode, disease determination, treatment guidance, efficacy prediction, and prognosis. However, the cost is high and a false negative result occurs easily.

Due to a variety of disadvantages above-mentioned, a novel clinical method for detection of HCV antigen, particularly HCV envelope antigen, is urgently required. The method should have high specificity, low cost, rapid diagnosis and is easy for practice.

In recent years, the study of DNA aptamers opens a new channel for treatment of various diseases. As a reagent for early diagnosis and treatment of HCV, HCV-E2 DNA aptamer plays an important role in screening HCV of blood donors, determining an early infection, fighting against HCV infection, and treating hepatitis C. Furthermore, aptamers will replace antibody in some aspects and thereby develop into a novel receptor inhibitor and detection reagent.

SELEX (Systematic Evolution of Ligands by Exponential Enrichment) technology is a new combinatorial chemistry technology developed in the early 1990s. The principle of the technology is that a large amount of random oligonucleotide library is selected, amplified through PCR, specifically bound to target molecules, and screened repetitively to yield an aptamer having high affinity and specificity. The advantages of the technology include large library capacity, a wide range of target molecules, high affinity, and wide application. The method has been applied to screening of various target molecules including metal ions, organic dyes, proteins, drugs, amino acids, and a variety of cytokines. The method is simple, rapid, and economic. Compared with other combinational chemical libraries such as random peptide libraries, antibody libraries, and phage display libraries, aptamers screened from oligonucleotide libraries have much higher affinity and specificity, with good prospects. Compared with conventional antibody, aptamers have low molecular weight, penetrate into cells more quickly, and can be synthesized stably and removed quickly, and easy for modification. Therefore, it is very promising as a new reagent of prevention, diagnosis and treatment of diseases.

So, according to the above description, to screen small-molecule nucleotide aptamer against HCV by SELEX technology will lay the foundation for the study of HCV infection mechanism and the development of diagnostic reagents against HCV.

### SUMMARY OF THE INVENTION

In view of the above-described problems, it is one objective of the invention to provide a small-molecule nucleotide aptamer for hepatitis C virus (HCV) which functions as an antagonist for prevention and treatment of hepatitis C.

It is another objective of the invention to provide a method of preparing a small-molecule nucleotide aptamer against HCV which functions as an antagonist for prevention and treatment of hepatitis C.

It is still another objective of the invention to provide a pharmaceutical composition for prevention and treatment of hepatitis C.

It is further an objective of the invention to provide a diagnostic reagent for detection of HCV surface antigen.

It is still another objective of the invention to provide a method for detection of HCV infection.

In another aspect, the invention provides a method of prevention and treatment of HCV infection.

To achieve the above objectives, in accordance with one embodiment of the invention, provided is a DNA aptamer against HCV comprising a nucleotide sequence as shown in SEQIDNO.1, SEQIDNO.2, SEQIDNO.3, SEQIDNO.4, SEQIDNO.5, SEQIDNO.6, SEQIDNO.7, SEQIDNO.8, SEQIDNO.9, SEQIDNO.11, SEQIDNO.12, SEQIDNO.13, SEQIDNO.14, SEQIDNO.15, SEQIDNO.16, SEQIDNO.17, SEQIDNO.18, SEQIDNO.19, SEQIDNO.20, SEQIDNO.21, SEQIDNO.22, SEQIDNO.23, SEQIDNO.24, SEQIDNO.25, SEQIDNO.26, SEQIDNO.27, SEQIDNO.28, and SEQIDNO.29.

In accordance with another embodiment of the invention, provided is a method of preparing a small-molecule nucleotide aptamer against HCV which functions as an antagonist for prevention and treatment of hepatitis C, the method comprising the steps of:
a) constructing a single-stranded DNA (ssDNA) library (88 base), 5′-GCGGAATTCTAATACGACTCACTATAGGGAACAGTCCGA GCC-N₃₀-GGGTCAATGCGTCATA-3′, an upstream primer, 5′-GCGGAATTCTAATACGACTCACTATAGGGAACAGTCCGAGCC-3′, and a downstream primer, 5′-GCGGGATCCTATGACGCATTGACCC-3′, wherein N represents A, G, T, or C, the library capacity is between 10¹⁴ and 10¹⁵, the underlined part comprises a T7 promoter sequence, the upstream primer comprises an EcoRI restriction site, and the downstream primer comprises an BamHI restriction site; the single-stranded DNA library and primers can be purchased from a primer synthesis company (such as SBS Genetech Co., Ltd.);
b) amplifying the single-stranded DNA library into a double-stranded DNA (dsDNA) library (totally 14 cycles), conserving, and amplifying the double-stranded DNA library to yield another single-stranded DNA library for next screening, the reaction program for PCR being 94° C. 4 min, 94° C. 30 s, 56° C. 45 s, 72° C. 90 s, for 18-25 cycles, and then 72° C. 7 min; the best amplification effect being obtained by modifying the cycle number (18-25 cycles);
c) electrophoresing a product of PCR amplification from step b) with 2 g/100 mL agarose gel containing 0.5 μg/mL ethidium bromide, placing the resultant product on a 260 nm fluoroscopy board, cutting an orange stripe, and purifying the orange stripe with a DNA purification kit (manufactured by Qiagen Co., Ltd., German);
d) placing 8 μg of ssDNA aptamer from step c) in a bath at 85° C. for 15 min and in an ice bath for 5 min respectively, mixing with CT26-HCV-E2 (10⁸) in a 1× screening buffer, oscillating at 37° C. for 30 min, 2000 rpm for 5 min, removing supernatant, washing with 1× screening eluent for 4-6 times, centrifugating, collecting cells, blowing homogenously with 50 μL of sterile double-distilled water, boiling for 5 min, putting in an ice bath, extracting with phenol:chloroform=25:24, collecting supertanant, amplifying to yield a dsDNA library, performing single-stranded amplification with the dsDNA library as a template, and purifying by the method of step c) to yield ssDNA aptamer for next screening;
the screening buffer 2× is 25 mmol/L Tris-HCl buffer, 50 mmol/L KCl, 200 mmol/L NaCl, 0.2 mmol/L EDTA, 5 mL/100 mL of glycerol, or 0.5 mmol/L dithiothreitol (DTT);
the screening eluent 2× is 25 mmol/L Tris-HCl buffer, 50 mmol/L KCl, 1 mol/L NaCl, 0.2 mmol/L EDTA, 5% glycerol, or 0.5 mmol/L dithiothreitol (DTT);
e) repeating step d) for a second and a third round of screening with 10⁸ CT26-HCV-E2 (Li P F, et al., Vaccine, 25: 1544-1551), and the ssDNA ampamer obtained from the previous round is used for next round of screening;
f) collecting 8 μg of single-stranded DNA aptamer from the third round of screening, placing in a bath at 85° C. for 15 min and in an ice bath for 5 min respectively, mixing with 10⁶ CT26 (Li P F, et al., Vaccine, 25: 1544-1551) in a 1× screening buffer, oscillating at 37° C. for 30 min, 2000 rpm for 5 min, collecting supernatant, mixing with 10⁶ CT26-HCV-E2 in a 1× screening buffer, oscillating at 37° C. for 30 min, 2000 rpm for 5 min, washing with 1× screening eluent for 4-6 times, centrifugating, collecting cells, blowing homogenously with 50 μL of sterile double-distilled water, boiling for 5 min, putting in an ice bath, extracting with phenol:chloroform=25:24, collecting supertanant, amplifying to yield dsDNA library, and performing single-stranded amplification with the dsDNA library as a template to yield ssDNA aptamer for next screening;
g) repeating step f) for a fifth and a sixth round of screening, and the ssDNA ampamer obtained from the previous round is used for next round of screening; repeating step f) for a seventh, eighth, and ninth round of screening, and the CT26 is 10⁷, the CT26-HCV-E2 is 10⁶, the ssDNA ampamer obtained from the previous round is used for next round of screening; repeating step f) for a tenth to fourteenth round of screening, and the CT26 is 10⁸, the CT26-HCV-E2 is 10⁵, the ssDNA ampamer obtained from the previous round is used for next round of screening; and
h) comparing the affinity of each round of ssDNA with CT26-HCV-E2, amplifying an ssDNA aptamer having the highest affinity (the thirteenth round of aptamer) with CT26-HCV-E2 following the method of step b) to yield dsDNA, digesting with DNA endonuclease EcoRI and BamHI, connecting to plasmid pUC19 (Yanisch-Perron, C., et al., 1985), transforming into E. coli DH5α (Hanahan, D., 1983; Tartof, K. D., et al., 1987), screening with ampicillin, and sequencing screened single bacterial colony.

By the method, the obtained aptamers are SEQIDNO.1, SEQIDNO.2, SEQIDNO.3, SEQIDNO.4, SEQIDNO.5, SEQIDNO.6, SEQIDNO.7, SEQIDNO.8, SEQIDNO.9, SEQIDNO.11, SEQIDNO.12, SEQIDNO.13, SEQIDNO.14, SEQIDNO.15, SEQIDNO.16, SEQIDNO.17, SEQIDNO.18, SEQIDNO.19, SEQIDNO.20, SEQIDNO.21, SEQIDNO.22, SEQIDNO.23, SEQIDNO.24, SEQIDNO.25, SEQIDNO.26, SEQIDNO.27, SEQIDNO.28, and SEQIDNO.29 as shown in Sequence Listing.

The obtained small-molecule nucleotide aptamer can play the following roles described below for prevention or treatment of HCV infection.

1. The small-molecule nucleotide aptamer inhibits competitively the binding of the acceptor CD81 (Cao J, et al., et al., 2007, J Microbiol Methods, 68(3):601-4) to HCV (Zhong J, et al., 2005, Proc Natl Acad Sci USA, 102(26): 9294-9) antigen E2. CD81 is a receptor of HCV envelope glycoprotein E2, and can inhibit the binding of the aptamer to CT26-HCV-E2. 300 ng/100 μL purified CD81 and cells were incubated at 37° C. for 60 min, 2000 rpm, and the precipitated cells were washed with PBS thrice. 4 μg of FITC-labeled aptamer/100 μL was added, incubated, and washed following the method described above. A control group without CD81 was established. The fluorescence intensity was measured with a flow cytometry. The results showed CD81 inhibited the binding of both aptamer library and a single aptamer to HCV antigen E2, which meant CD81 competed with the aptamer to bind to E2. Different single aptamer has different binding site with E2. Therefore, the aptamer can be used as a medication interfering in the binding of HCV to acceptors in vivo.

2. Experiments of small-molecule nucleotide aptamer inhibiting the binding of HCV envelop antigen E2 to human liver cells

Human liver cancer cells Huh 7.5.1 have natural HCV acceptors, following the method described above, the similar results are obtained (the binding rate decreases from 36.7% to 15.4%), which means the aptamer can inhibit the binding of GST-E2 (Li P F, et al., Vaccine, 25: 1544-1551.) to Huh 7.5.1 (Zhong J, et al., 2005, Proc Natl Acad Sci USA, 102(26):9294-9). Further experiment showed that the inhibition exhibited dose-dependent and dose-saturated.

3. Application of small-molecule nucleotide aptamer as material for preparation of medication for prevention or treatment of HCV infection, i.e., experiments of small-molecule nucleotide aptamer inhibiting the infection of live HCV on human liver cells

1) Immunofluorescence

a) Huh 7.5.1 cells were cultured in a 96-well plate, 37° C. and 5% CO₂;

b) HCV (3×10⁵, 18 μL)+samples (8 μg and 4 μg of aptamer), 37° C. for an hour (three wells, 180 μL/well);

c) Huh 7.5.1 cells were washed with PBS, and the incubated virus were added, cultured at 37° C. and 5% CO₂ for 5 hours;

d) the cells were washed, added to a culture medium, and cultured at 37° C. and 5% CO₂ for 72 hours;

e) the cells were washed and monoclonal antibody HCV-E2 was added for further culture (Zhong J, et al., 2005, Proc Natl Acad Sci USA, 102(26):9294-9); and

f) the cells were washed and red fluorescence points of each well were counted under a fluorescence microscope (ffu/well, 580 nm).

2) Fluorescent Real-Time Quantitative RT-PCR Method

QuantiTect SYBR Green PCR Handbook Kit (manufactured by QIAGEN Co., Ltd) was used to quantifying HCV RNA of cells. Huh 7.5.1 cells were cultured in a 6-well plate, 4.5×10⁵/well, and aptamers, mutants thereof having different concentration, or 500 U IFN-α was added. 200 μL of JFH1-HCVcc (the content of virus was 10⁷ copies) was further added. The resultant plate was culture overnight at 37° C. The supernatant was removed. The cells were washed with DEPC-treated PBS, and the total RNA was extracted with TRIzol (manufactured by Invitrogen Life Technologies Co., Ltd.). The RNA (the total volume 20 μL) was transcripted reversly with First Strand cDNA synthesis kit (manufactured by Fergment Co., Ltd.), at presence of 0.5 μg oligo(dT)18 as a primer, 1 μL of RNase inhibitor, 1 μL of M-MLV reverse transcriptase, and 2 μL of 10×RT buffer (manufactured by Ambion Co., Ltd.), firstly 42° C. for 45 min, and then 75° C. for 10 min to synthesize cDNA. The upstream and downstream primers for HCV amplification were 5′AATGGCTCGAGGAAACTGTGAAGCGA3′ and 5′TTCATCATGCCAATGGTGTTCGTGGC3′ respectively. The PCR program was: 94° C. for 5 min, 95° C. for 10 s, 58° C. for 20 s, and 72° C. for 30 s, totally 45 cycles. The results were analyzed by Rotogene software.

3) Western Blot Method

Huh 7.5.1 cells were cultured in a 6-well plate, 4.5×10⁵/well, and aptamers, mutants thereof having different concentration, or 500 U IFN-α was added. 200 μL of JFH1-HCVcc (the content of virus was 10⁷ copies) was further added. The resultant plate was culture overnight at 37° C. The cells were dissolved in a 200 μL of SDS-loading buffer at 100° C. for 5 min and electrophoresed at 12% SDS-polyacrylamide gel solution. The obtained proteins were transferred to a PVDF membrane. HCV-E2 was measured by anti-E2 antibody. β-actin (internal reference) was measured by anti-β-actin antibody.

4. Cytotoxicity Assay of Aptamers

a) Huh 7.5.1 cells were cultured in a 96-well plate, about 3×10³ cells/well;

b) after the cells were attached to the wall, aptamers having different concentration were added, 6 wells for each concentration;

c) 72 hours later, the supertanant was removed, 80 μL new medium was added, 20 μL of 5 mg/mL MTT was further added to each well and cultured for 4 hours;

d) the supertanant was removed and 150 μL of DMSO was added, mixing, and shaking for 10 min to make crystal dissolved completely; and

e) OD₅₇₀ was measured by an ELISA reader to calculate IC₅₀.

Inhibition rate=((control−blank)−(sample−blank))/(control−blank)×100%

1 gIC50=Xm−I(P−(3−Pm−Pn)/4), wherein Xm represents 1 g(maximum dose), I represents 1 g(maximum dose/adjacent dose), P represents the summation of positive response rate, Pm represents maximum positive response rate, and Pn represents minimum positive response rate.

The measured IC₅₀ of single aptamer=3.35×104 μg/100 μL=10.47 mmol/L.

Advantages of the invention are summarized below:
1) The aptamers of the invention can significantly inhibit HCV infection on cells by binding to HCV envelop glycoprotein E2. The aptamers have low toxicity, can be used directly as an antagonist against HCV for detection, prevention, and treatment of hepatitis C. That the aptamers of the invention is screened with SELEX technology ensures the aptamers can bind to HCV active site, and thereby HCV can not bind to CD81, can not enter a host cell, and can not stay and multiply in vivo, all of which benefit the immune system to eliminate the virus.
2) The aptamers of invention provide effective and powerful means for early and sensitive detection of HCV. HCV is mainly spread by blood-to-blood contact. The infection is often asymptomatic, but once established, chronic infection can progress to scarring of the liver (fibrosis), and advanced scarring (cirrhosis). In some cases, those with cirrhosis will go on to develop liver cancer. No vaccine against hepatitis C is available to date. Therefore, to detect HCV accurately and sensitively in blood source before transfusion is a key step for prevention of HCV infection. ELISA has been widely used for detection HCV antibody to determine whether an infection occurs. However, during the early HCV infection, or for a patient with immunodeficiency syndrome, no antibody produced even there is an HCV infection. Furthermore, by ELISA, a false positive or false negative result may be obtained. As another assistant method for detection of HCV infection, RT-PCR is costly, cause pollution easily, so it is not suitable for clinical application. Therefore, DNA aptamers are a better diagnostic reagent for early detection of HCV than antibody.
3) The aptamers of the invention are small-molecule nucleotide, with different molecular structure compared with any other broad-spectrum antibiotic, so there is no question about its resistance. Additionally, DNA aptamers of the invention are specific to HCV, cause no harm to a variety of beneficial bacteria and cells in vivo. Compared with protein antibody, DNA aptamers have small molecular weight, penetrate into cells quickly, no antigenicity, and cause no side effect.
4) The aptamers (libraries) of the invention have been cloned to plasmid pUC19 which has been transformed to E. Coli DH5α, so the aptamers can be produced in large scale by the bacteria. The aptamers can also be synthesized directly.

### BRIEF DESCRIPTION OF THE DRAWINGS

The invention is described hereinbelow with reference to accompanying drawings, in which:

FIG. 1 shows an establishment of stable cell line CT26-HCV-E2 which expresses protein HCV-E2 according to one embodiment of the invention; there are more protein E2 at the surface of cells CT26-HCV-E2 than that of CT26, and E2-CT26 can be used as target cells for screening HCV-E2 aptamers; A: protein E2 expressed at the surface of cells CT26-HCV-E2; B: protein E2 expressed in the cytoplasm of cells CT26-HCV-E2;

FIG. 2 is a flow chart of screening specific aptamers against HCV with CELL-SELEX technology according to one embodiment of the invention; randomly synthesized single-stranded oligonucleotide libraries are mixed with cells CT26-HCV-E2, unbound aptamers are removed, after three rounds of screening, cells CT26 are added for negative screening, there are totally 14 rounds of screening; finally, aptamers which can bind to E2-CT26 and not bind to CT26 are screened by SELEX;

FIG. 3 is a schematic diagram of amplification of single-stranded and double-stranded DNA according to one embodiment of the invention; before each round of screening, an ssDNA library are amplified into a dsDNA library, conserved, and the obtained dsDNA is further amplified into another ssDNA library for next screening; the figure shows an electrophoretic mobility of ssDNA and dsDNA, and after PCR, the aptamers are used for screening (M: Marker; 1-5: ssDNA; 6-10: dsDNA);

FIG. 4 shows an binding capacity of ssDNA aptamer library with cells CT26-HCV-E2 according to one embodiment of the invention; each round of screened ssDNA (8 μg) are mixed with 10⁶ CT26-HCV-E2 respectively, and the results show the thirteenth round of aptamer library has the strongest binding capacity with the cells, and the binding is dose-dependent; A: the thirteenth round of aptamer library has the strongest binding capacity (89%); B: the binding capacity of a single aptamer cloned from the thirteenth round of aptamer library with E2-CT26 is dose-dependent;

FIG. 5 shows the receptor CD81 of HCV-E2 can inhibit the binding of the screened aptamer libraries (the thirteenth and the twelfth) and a single aptamer with protein E2 according to one embodiment of the invention; CD81 is a receptor of HCV envelope glycoprotein E2, and can inhibit the binding of the aptamer to CT26-HCV-E2; 300 ng/100 μL purified CD81 and cells are incubated, and then 4 μg of FITC-labeled aptamer/100 μL is added; a control group without CD81 is established; the results showed CD81 inhibits the binding of both aptamer library (for the thirteenth library, the binding rate decreases from 10.2% to 7.6%) and a single aptamer (the binding rate decreases from 14.8% to 5.8%), particularly for a single aptamer; the figure shows the screened aptamer libraries (the thirteenth library and the twelfth library) and the single aptamer can inhibit the binding of HCV-E2 to an acceptor thereof; 4thP: the fourth round of screened library; 12thP: the twelfth round of screened library; 13thP: the thirteenth round of screened library; the single aptamer is cloned from the thirteenth round of screened library;

FIG. 6 shows aptamers inhibit the binding of HCV-E2 to human liver cells according to one embodiment of the invention; human liver cancer cells Huh 7.5.1 have born HCV acceptors, and the binding rate of the cells to protein GST is 1%, to protein E2 36.7%; after addition of the thirteenth round of aptamer, the binding rate decreases to 23.2%, and after addition of a single aptamer, the binding rate decreases to 15.4%, which means that the aptamer can inhibit the binding of HCV to an acceptor thereof; 1stP: the first round of screened library; 6thP: the sixth round of screened library; 13thP: the thirteenth round of screened library;

FIG. 7 shows aptamers inhibit the infection of live HCV on liver cells according to one embodiment of the invention, and the inhibition is dose-dependent; 7A: a single aptamer inhibits the infection of HCV JFH-1 on liver cell Huh 7.5.1, and the infection is dose-dependent, H represents a high dose, L represents a low dose, and the result is obtained by an immunofluorescence microscope; 7B: an aptamer inhibits the infection of HCVcc on liver cell Huh 7.5.1 by fluorescent real-time quantitative RT-PCR method, and the infection is dose-dependent; 7C: an aptamer inhibits the infection of HCVcc on liver cell Huh 7.5.1 by Western blot method, and the infection is dose-dependent; 7D: an aptamer inhibits the infection of HCVcc on liver cell Huh 7.5.1 with an immune confocal microscope, while an mutant of the aptamer has no obvious inhibition capacity; and

FIG. 8 shows a result of cytotoxic assay of an aptamer according to one embodiment of the invention, and IC₅₀=3.35×104 μg/100 μL=10.47 mmol/L.

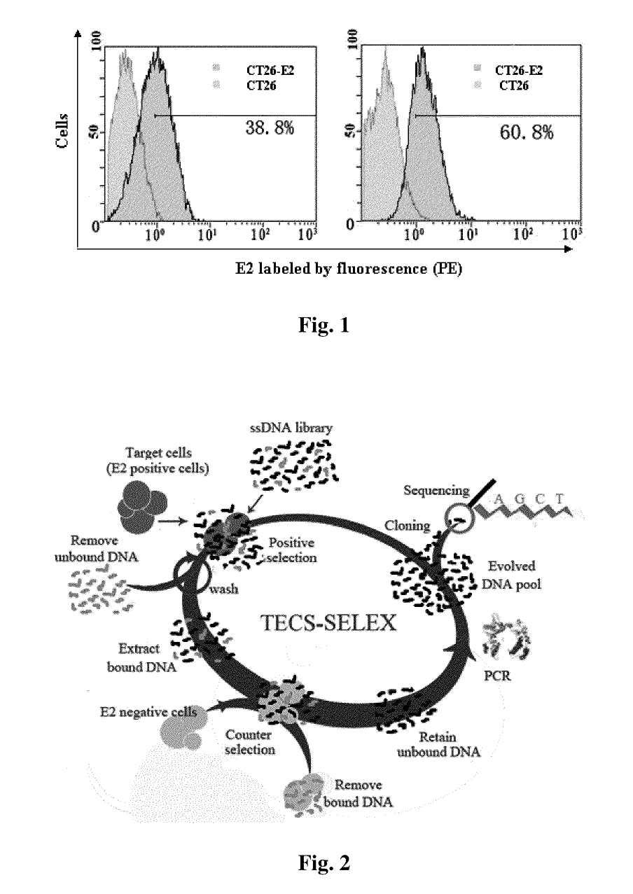

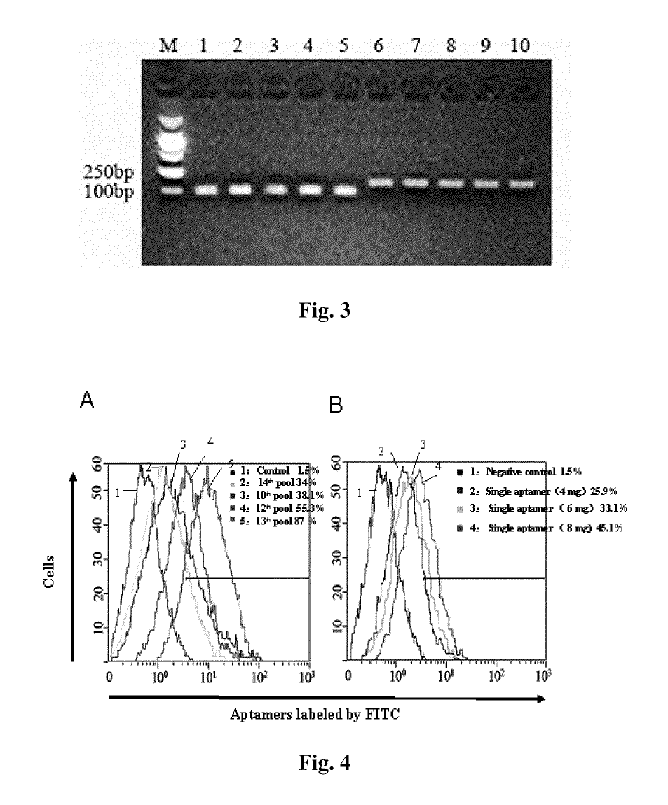

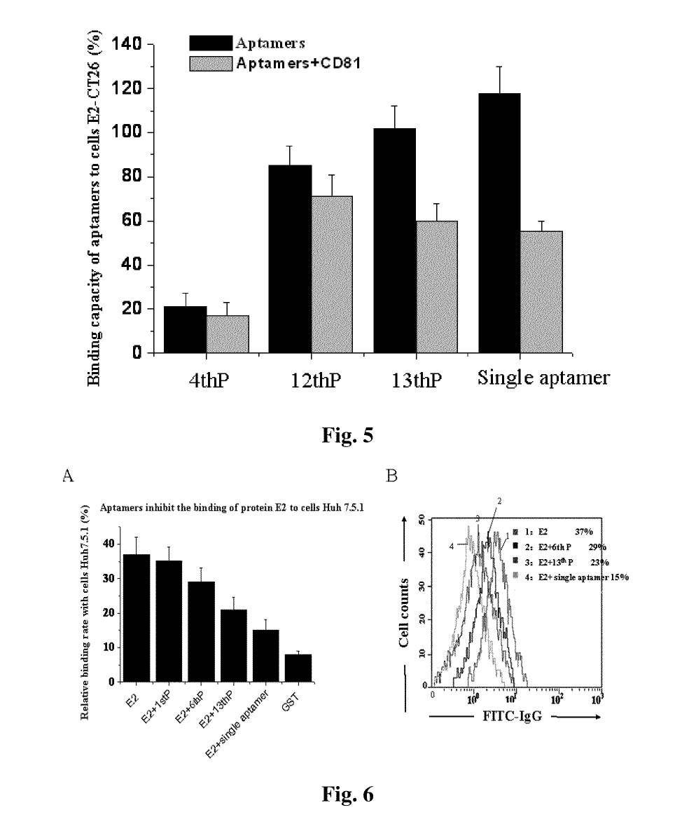

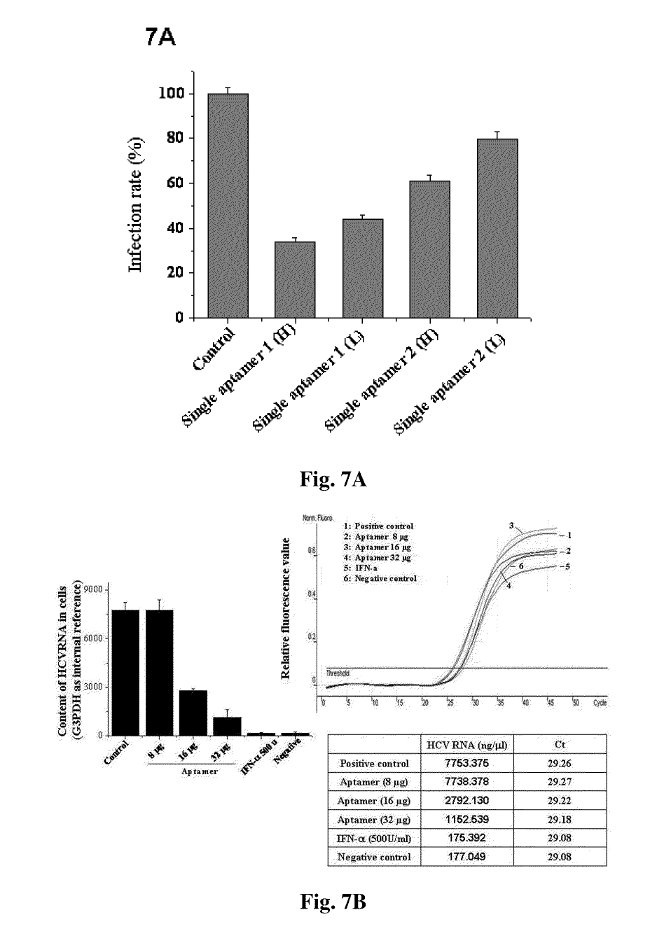

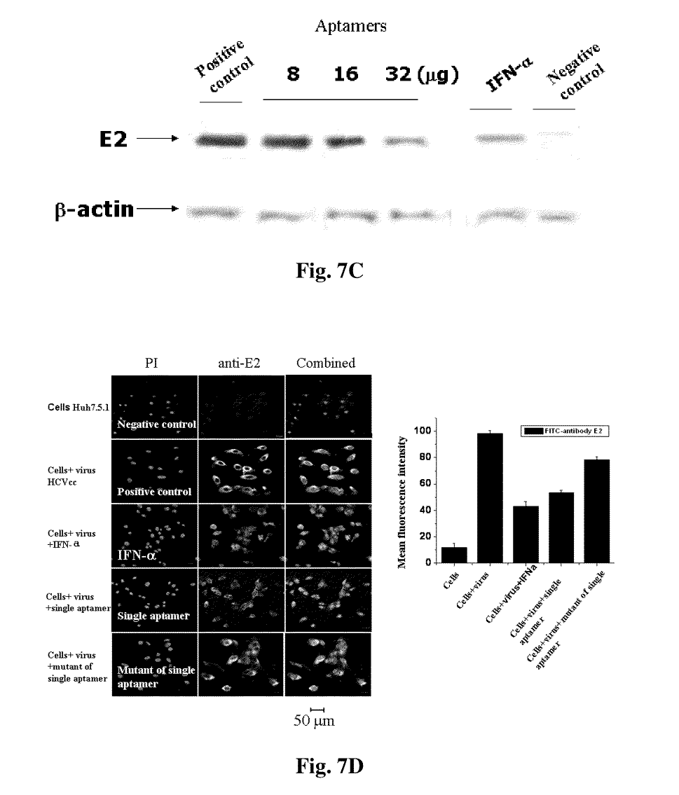

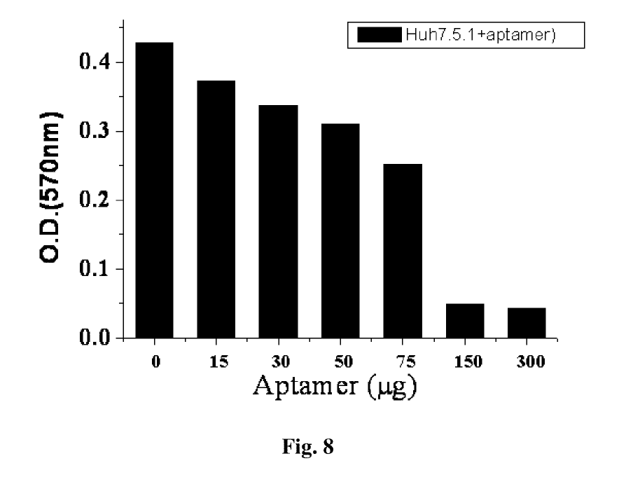

### DETAILED DESCRIPTION OF THE EMBODIMENTS

For further illustrating the invention, embodiments detailing a small-molecule nucleotide sequence for hepatitis C virus (HCV), preparation method and method of use thereof are described below. It should be noted that the following embodiments are intended to describe and not to limit the invention.

In the invention, a DNA aptamer against HCV comprising a nucleotide sequence as shown in SEQIDNO.1-29 is constructed.

Secondly, provided is a method of preparing a small-molecule nucleotide aptamer against HCV which functions as an antagonist for prevention and treatment of hepatitis C, the method comprising the steps of:
a) constructing a single-stranded DNA (ssDNA) library (88 base), 5′-GCGGAATTCTAATACGACTCACTATAGGGAACAGTCCGA GCC-N₃₀-GGGTCAATGCGTCATA-3′, an upstream primer, 5′-GCGGAATTCTAATACGACTCACTATAGGGAACAGTCCGAGCC-3′, and a downstream primer, 5′-GCGGGATCCTATGACGCATTGACCC-3′, wherein N represents A, G, T, or C, the library capacity is between 10¹⁴ and 10¹⁵, the underlined part comprises a T7 promoter sequence, the upstream primer comprises an EcoRI restriction site, and the downstream primer comprises an BamHI restriction site; the single-stranded DNA library and primers can be purchased from Shanghai Bioengineering Company;
b) amplifying the single-stranded DNA library into a double-stranded DNA (dsDNA) library (totally 14 cycles), conserving, and amplifying the double-stranded DNA library to yield another single-stranded DNA library for next screening, the reaction program for PCR being 94° C. 4 min, 94° C. 30 s, 56° C. 45 s, 72° C. 90 s, for 18-25 cycles, and then 72° C. 7 min; the best amplification effect being obtained by modifying the cycle number (18-25 cycles);
c) electrophoresing a product of PCR amplification from step b) with 2 g/100 mL agarose gel containing 0.5 μg/mL ethidium bromide, placing the resultant product on a 260 nm fluoroscopy board, cutting an orange stripe, and purifying the orange stripe with a DNA purification kit; the purification kit being purchased from Qiagen Company, German;
d) placing 8 μg of ssDNA aptamer from step c) in a bath at 85° C. for 15 min and in an ice bath for 5 min respectively, mixing with CT26-HCV-E2 (10⁸) in a 1× screening buffer, oscillating at 37° C. for 30 min, 2000 rpm for 5 min, removing supernatant, washing with 1× screening eluent for 4-6 times, centrifugating, collecting cells, blowing homogenously with 50 μL of sterile double-distilled water, boiling for 5 min, putting in an ice bath, extracting with phenol:chloroform=25:24, collecting supertanant, amplifying to yield a dsDNA library, performing single-stranded amplification with the dsDNA library as a template, and purifying by the method of step c) to yield ssDNA aptamer for next screening;
the screening buffer 2× is 25 mmol/L Tris-HCl buffer, 50 mmol/L KCl, 200 mmol/L NaCl, 0.2 mmol/L EDTA, 5 mL/100 mL of glycerol, or 0.5 mmol/L dithiothreitol (DTT);
the screening eluent 2× is 25 mmol/L Tris-HCl buffer, 50 mmol/L KCl, 1 mmol/L NaCl, 0.2 mmol/L EDTA, 5% glycerol, or 0.5 mmol/L dithiothreitol (DTT);
e) repeating step d) for a second and a third round of screening with 10⁸ CT26-HCV-E2, and the ssDNA ampamer obtained from the previous round is used for next round of screening;
f) collecting 8 μg of single-stranded DNA aptamer from the third round of screening, placing in a bath at 85° C. for 15 min and in an ice bath for 5 min respectively, mixing with 10⁶ CT26 in a 1× screening buffer, oscillating at 37° C. for 30 min, 2000 rpm for 5 min, collecting supernatant, mixing with 10⁶ CT26-HCV-E2 in a 1× screening buffer, oscillating at 37° C. for 30 min, 2000 rpm for 5 min, washing with 1× screening eluent for 4-6 times, centrifugating, collecting cells, blowing homogenously with 50 μL of sterile double-distilled water, boiling for 5 min, putting in an ice bath, extracting with phenol: chloroform=25:24, collecting supertanant, amplifying to yield dsDNA library, and performing single-stranded amplification with the dsDNA library as a template to yield ssDNA aptamer for next screening;
g) repeating step f) for a fifth and a sixth round of screening, and the ssDNA ampamer obtained from the previous round is used for next round of screening; repeating step f) for a seventh, eighth, and ninth round of screening, and the CT26 is 10⁷, the CT26-HCV-E2 is 10⁶, the ssDNA ampamer obtained from the previous round is used for next round of screening; repeating step f) for a tenth to fourteenth round of screening, and the CT26 is 10⁸, the CT26-HCV-E2 is 10⁵, the ssDNA ampamer obtained from the previous round is used for next round of screening; and
h) comparing the affinity of each round of ssDNA with CT26-HCV-E2, amplifying an ssDNA aptamer having the highest affinity (the thirteenth round of aptamer) with CT26-HCV-E2 following the method of step b) to yield dsDNA, digesting with DNA endonuclease EcoRI and BamHI, connecting to plasmid pUC19 (Yanisch-Perron, C., et al., 1985), transforming into E. coli DH5α(Hanahan, D., 1983; Tartof, K. D., et al., 1987), screening with ampicillin, and sequencing screened single bacterial colony.

The obtained small-molecule nucleotide aptamer can play the following role described below for prevention or treatment of HCV infection.

1. The small-molecule nucleotide aptamer inhibits competitively the binding of the receptor CD81 to HCV antigen E2. CD81 is a receptor of HCV envelope glycoprotein E2, and can inhibit the binding of the aptamer to CT26-HCV-E2. 300 ng/100 μL purified CD81 and cells were incubated at 37° C. for 60 min, 2000 rpm, and the precipitated cells were washed with PBS thrice. 4 μg of FITC-labeled aptamer/100 μL was added, incubated, and washed following the method described above. A control group without CD81 was established. The fluorescence intensity was measured with a flow cytometry. The results showed CD81 inhibited the binding of both aptamer library and a single aptamer (ZE18) to HCV antigen E2, particularly ZE18, but the inhibition on single aptamers ZE14 and ZE25 was not so significant, which meant CD81 competed with the aptamer to bind to E2, and different single aptamer has different binding site with E2. Therefore, the aptamer can be used as a medication interfering in the binding of HCV to acceptors in vivo.

2. Experiments of small-molecule nucleotide aptamer inhibiting the binding of HCV envelop antigen E2 to human liver cells

Human liver cancer cells Huh 7.5.1 have born HCV acceptors, following the method described above, the similar results are obtained (the binding rate decreases from 36.7% to 15.4%), which means the aptamer can inhibit the binding of GST-E2 to Huh 7.5.1. Further experiment showed that the inhibition exhibited dose-dependent and dose-saturated.

3. Application of small-molecule nucleotide aptamer as material for preparation of medication for prevention or treatment of HCV infection, i.e., experiments of small-molecule nucleotide aptamer inhibiting the infection of live HCV on human liver cells

1) Immunofluorescence

a) Huh 7.5.1 cells were cultured in a 96-well plate, 37° C. and 5% CO₂;

b) HCV (3×10⁵, 18 μL)+samples (8 μg and 4 μg of aptamer), 37° C. for an hour (three wells, 180 μL/well);

c) Huh 7.5.1 cells were washed with PBS, and the incubated virus were added, cultured at 37° C. and 5% CO₂ for 5 hours;

d) the cells were washed, added to a culture medium, and cultured at 37° C. and 5% CO₂ for 72 hours;

e) the cells were washed and monoclonal antibody PE-E2 was added for further culture (Zhong J, et al., 2005, Proc Natl Acad Sci USA, 102(26):9294-9); and

f) the cells were washed and red fluorescence points of each well were counted under a fluorescence microscope (ffu/well, 580 nm).

2) Fluorescent Real-Time Quantitative RT-PCR Method

QuantiTect SYBR Green PCR Handbook Kit (manufactured by QIAGEN Co., Ltd.) was used to quantifying HCV RNA of cells. Huh 7.5.1 cells were cultured in a 6-well plate, 4.5×10⁵/well, and aptamers, mutants thereof having different concentration (4 μg/100 μL, 8 μg/100 μL, 16 μg/100 μL, and the mutants mutated by 2 base), or 500 U IFN-α was added. 200 μL of JFH1-HCVcc (the content of virus was 10⁷ copies) was further added. The resultant plate was culture overnight at 37° C. The supernatant was removed. The cells were washed with DEPC-treated PBS, and the total RNA was extracted with TRIzol (manufactured by Invitrogen Life Technologies Co., Ltd.). The RNA (the total volume 20 μL) was transcripted reversly with First Strand cDNA synthesis kit (manufactured by Fergment Co., Ltd.), at presence of 0.5 μg oligo(dT)18 as a primer, 1 μL of RNase inhibitor, 1 μL of M-MLV reverse transcriptase, and 2 μL of 10×RT buffer (manufactured by Ambion Co., Ltd.), firstly 42° C. for 45 min, and then 75° C. for 10 min to synthesize cDNA. The upstream and downstream primers for HCV amplification were 5′AATGGCTCGAGGAAACTGTGAAGCGA3′ and 5′TTCATCATGCCAATGGTGTTCGTGGC3′ respectively. The PCR program was: 94° C. for 5 min, 95° C. for 10 s, 58° C. for 20 s, and 72° C. for 30 s, totally 45 cycles. The results were analyzed by Rotogene software.

3) Western Blot Method

Huh 7.5.1 cells were cultured in a 6-well plate, 4.5×10⁵/well, and aptamers, mutants thereof having different concentration, or 500 U IFN-α was added. 200 μL of JFH1-HCVcc (the content of virus was 10⁷ copies) was further added. The resultant plate was culture overnight at 37° C. The cells were dissolved in a 200 μL of SDS-loading buffer at 100° C. for 5 min and electrophoresed at 12% SDS-polyacrylamide gel solution. The obtained proteins were transferred to a PVDF membrane. HCV-E2 was measured by anti-E2 antibody. β-actin (internal reference) was measured by anti-β-actin antibody.

4. Cytotoxicity Assay of Aptamers

a) Huh 7.5.1 cells were cultured in a 96-well plate, about 3×10³ cells/well;

b) after the cells were attached to the wall, aptamers having 8 different of concentration (0.5-100 μg/100 μL) were added, 6 wells for each concentration;

c) 72 hours later, 20 μL of 5 mg/mL MTT was further added to each well and cultured for 4 hours;

d) the supertanant was removed and 100 μL of DMSO was added to terminate the reaction; and

e) OD₅₇₀ was measured by an ELISA reader to calculate IC₅₀.

Inhibition rate=((control−blank)−(sample−blank))/(control−blank)×100%

1 gIC50=Xm−I(P−(3−Pm−Pn)/4), wherein Xm represents 1 g(maximum dose), I represents 1 g(maximum dose/adjacent dose), P represents the summation of positive response rate, Pm represents maximum positive response rate, and Pn represents minimum positive response rate.

The measured IC50 of single aptamer=3.35×104 μg/100 μL=10.47 mmol/L.

While particular embodiments of the invention have been shown and described, it will be obvious to those skilled in the art that changes and modifications may be made without departing from the invention in its broader aspects, and therefore, the aim in the appended claims is to cover all such changes and modifications as fall within the true spirit and scope of the invention.

The invention claimed is:

1. A small-molecule nucleotide aptamer for hepatitis C virus comprising a nucleotide sequence as shown in SEQIDNO.1, SEQIDNO.2, SEQIDNO.3, SEQIDNO.4, SEQIDNO.5, SEQIDNO.6, SEQIDNO.7, SEQIDNO.8, SEQIDNO.9, SEQIDNO.10, SEQIDNO.11, SEQIDNO.12, SEQIDNO.13, SEQIDNO.14, SEQIDNO.15, SEQIDNO.16, SEQIDNO.17, SEQIDNO.18, SEQIDNO.19, SEQIDNO.20, SEQIDNO.21, SEQIDNO.22, SEQIDNO.23, SEQIDNO.24, SEQIDNO.25, SEQIDNO.26, SEQIDNO.27, SEQIDNO.28, or SEQIDNO.29.

2. A method of preparation of the small-molecule nucleotide aptamer of claim 1, comprising the steps of:
a) constructing a single-stranded DNA library, 5′-GCGGAATTCTAATACGACTCACTATAGGGAACAGTCCGA GCC-N30-GGGTCAATGCGTCATA-3′, an upstream primer, 5′-GCGGAATTCTAATACGACTCACTATAGGGAACAGTCCGAGC C-3′, and a downstream primer, 5′-GCGGGATCCTATGACGCATTGACCC-3′;
b) amplifying said single-stranded DNA library into a double-stranded DNA library, conserving, and amplifying said double-stranded DNA library to yield another single-stranded DNA library for next screening, for PCR, said single-stranded DNA library being 2 μL, said upstream primer 0.1 nmol, said downstream primer 0.1 nmol, 25 mmol/L MgCl₂ 6 μL, 2 mmol/L dNTP 10 μL, 10×PCR buffer 10 μL, DNA polymerase 2.5 U, and double-distilled water added to make total volume up to 100 μL, the PCR program being 94° C. 4 min, 94° C. 30 s, 56° C. 45 s, 72° C. 90 s, for 18-25 cycles, and then 72° C. 7 min;
c) electrophoresing a product of PCR amplification from step b) with 2% agarose gel containing 0.5 μg/mL ethidium bromide, placing the resultant product on a 260 nm fluoroscopy board, cutting an orange stripe, and purifying said orange stripe with a DNA purification kit;
d) placing 8 μg of single-stranded DNA aptamer from step c) in a bath at 85° C. for 15 min and in an ice bath for 5 min respectively, mixing with 10⁸ CT26-HCV-E2 in a 1× screening buffer, oscillating at 37° C. for 30 min, 2000 rpm for 5 min, removing supernatant, washing with 1× screening eluent for 4-6 times, centrifugating, collecting cells, blowing homogenously with 50 μL of sterile double-distilled water, boiling for 5 min, putting in an ice bath, extracting with phenol:chloroform=25:24, collecting supertanant, amplifying to yield dsDNA library, performing single-stranded amplification with said dsDNA library as a template, and purifying by the method of step c) to yield ssDNA aptamer for next screening;
e) repeating step d) for a second and a third round of screening, and the ssDNA ampamer obtained from the previous round is used for next round of screening;
f) collecting 8 μg of single-stranded DNA aptamer from said third round of screening, placing in a bath at 85° C. for 15 min and in an ice bath for 5 min respectively, mixing with 10⁶ CT26 in a 1× screening buffer, oscillating at 37° C. for 30 min, 2000 rpm for 5 min, collecting supernatant, mixing with 10⁶ CT26-HCV-E2 in a 1× screening buffer, oscillating at 37° C. for 30 min, 2000 rpm for 5 min, washing with 1× screening eluent for 4-6 times, centrifugating, collecting cells, blowing homogenously with 50 μL of sterileZ double-distilled water, boiling for 5 min, putting in an ice bath, extracting with phenol:chloroform=25:24, collecting supertanant, amplifying to yield dsDNA library, and performing single-stranded amplification with said dsDNA library as a template to yield ssDNA aptamer for next screening;
g) repeating step f) for a fifth and a sixth round of screening, and the ssDNA ampamer obtained from the previous round is used for next round of screening;
h) repeating step f) for a seventh, eighth, and ninth round of screening, and said CT26 is 10⁷, said CT26-HCV-E2 is 10⁶, the ssDNA ampamer obtained from the previous round is used for next round of screening;
i) repeating step f) for a tenth to fourteenth round of screening, and said CT26 is 10⁸, said CT26-HCV-E2 is 10⁵, the ssDNA ampamer obtained from the previous round is used for next round of screening; and
j) comparing the affinity of each round of ssDNA with CT26-HCV-E2, amplifying an ssDNA aptamer having the highest affinity with CT26-HCV-E2 following step b) to yield dsDNA, digesting with DNA endonuclease EcoRI and BamHI, connecting to plasmid pUC19, transforming into E. coli DH5α, screening with ampicillin, and sequencing selected single bacterial colony.

3. The method of claim 2, wherein in step f), CT26-HCV-E2 is used for positive screening and CT26 is for negative screening.

4. The method of claim 2, wherein in step d), said screening buffer 2× is 25 mmol/L Tris-HCl buffer, 50 mmol/L KCl, 200 mmol/L NaCl, 0.2 mmol/L EDTA, 5% glycerol, or 0.5 mmol/L dithiothreitol, and said screening eluent 2× is 25 mmol/L Tris-HCl buffer, 50 mmol/L KCl, 1 mmol/L NaCl, 0.2 mmol/L EDTA, 5% glycerol, or 0.5 mmol/L dithiothreitol; and said centrifugating is 12,000 rmp for 5 min.

5. A pharmaceutical composition for prevention or treatment of hepatitis C virus infection, comprising at least a small-molecule nucleotide aptamer of claim 1.

6. A method for prevention or treatment of hepatitis C virus infection comprising administering to a patient in need thereof a pharmaceutical composition of claim 5.
